(19) United States
(12) Patent Application Publication
(10) Pub. No.: US 2013/0005720 A1
(43) Pub. Date: Jan. 3, 2013

(54) PHARMACEUTICAL COMPOSITION FOR TREATING CANCER AND APPLICATIONS THEREOF

(75) Inventors: Ta-Chau CHANG, Taipei City (TW); Zi-Fu Wang, Taipei County (TW); Chih-Chien Cho, Taipei County (TW); Wei-Chun Huang, Taipei City (TW); Jen-Fei Chu, Taipei City (TW)

(73) Assignee: Academia Sinica, Taipei City (TW)

(21) Appl. No.: 13/172,975

(22) Filed: Jun. 30, 2011

(51) Int. Cl. A61K 31/5383; C12N 5/09; A61P 35/02; A61K 31/473; A61K 31/4745; A61P 35/00; A61K 31/4545; C12N 5/071

(52) U.S. Cl. 514/229.5; 514/318; 435/375; 514/297; 514/280

(57) ABSTRACT

This invention relates to a pharmaceutical composition for treating cancer, comprising an effective amount of a compound represented by formula (I):

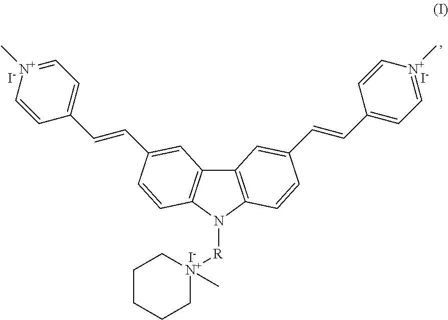

wherein R is —CCOCCOCC— or —CCOCCOCCOCC—; and a pharmaceutical acceptable carrier. This invention also relates to a method for treating cancer, comprising administering a therapeutically effective amount of said compound to a subject; and a method for stabilizing G4 structure of telomere by using this compound.

### BACKGROUND OF THE INVENTION

1. Field of the Invention

The present invention relates to a pharmaceutical composition for treating cancer, comprising an effective amount of a compound represented by formula (I). It also relates to a method for treating cancer and a method for forming a stabilized G-quadruplex structure by using the same.

2. Description of the Related Art

Telomeres play a vital role in protecting the ends of chromosomes and preventing chromosomal fusion (see Blackburn et al., 1996; Blasco, 2005; Cech, 2004). It has been known that a short 3′-overhang of many hexameric repeats of TTAGGG single-stranded sequence could adopt an intramolecular G-quadruplex (G4) structure under physiological conditions both in vitro (see Williamson, 1994; Wang et al., 1993; Parkinson et al., 2002) and in vivo (see Chang et al., 2004a; Maizels, 2006).

G4 structure is a four-stranded structure formed by a guanine (G)-rich DNA or RNA sequence, such as the sequence of chromosomal telomere. It is demonstrated that the folding of telomeric DNA into G4 structures is important in inhibiting the activity of telomerase, so the length of telomere cannot be maintained, the cell mitosis is stopped, and the cancer cells go through apoptosis (see Zahler et al., 1991; Bodnar et al., 1998). Therefore, G4 structure can be a potential target for cancer therapeutic intervention (see Mergny et al., 1998; Hurley, 2002; Neidle et al., 2003). In addition, small molecules that can induce structural changes or stabilize G4 structures of human telomere have the potential to act as anticancer agents. During the last few years, significant progress in the design of small molecules for targeting the G4 structures has been reported (see DeCian et al., 2008; Monchaud et al., 2008; Reed et al., 2006; DeCian et al., 2007; Müller et al., 2009).

Since the intracellular environment is highly crowded with various biomolecules, the molecular crowding condition has been introduced to mimic the physiological condition in living cells (see Miyoshi et al., 2008; Miyoshi et al., 2002). It has been found that PEG (polyethylene glycol) can induce G4 formation of human telomere under a salt-deficient condition in molecular crowding experiments. Moreover, PEG can convert the conformation of G4 structures from antiparallel to parallel (see Kan et al., 2006; Zhou et al., 2008). It shows that the oxygen atoms of PEG can result in dehydration for structural change (see Miyoshi et al., 2006; Xue et al., 2007). Recently, both NMR (Martadinata et al., 2009) and X-ray (Collie et al., 2010) analyses show the formation of parallel G4 structures from the telomere RNA in the presence of K⁺. Regarding with DNA, although X-ray result shows that telomeric DNA forms a similar parallel G4 structure (see Parkinson et al., 2002), NMR analysis shows that telomeric DNA forms a nonparallel G4 structures in a K⁺ solution (see Ambrus et al., 2006; Luu et al., 2006; Lim et al., 2009). The major difference between RNA and DNA is that the hydroxyl group in the sugar moiety of RNA is substituted by the hydrogen in the sugar moiety of DNA, which implies that the oxygen plays an important role in telomere G4 structures.

### SUMMARY OF THE INVENTION

The objects of the present invention is to design a novel stabilizer for G4 structure, which is able to highly stabilize the G4 structure of telomere in human chromosome and can be used as a drug for cancer therapeutic intervention.

To achieve the objects, the present invention provides a pharmaceutical composition for treating cancer, comprising a therapeutically effective amount of a compound represented by formula (I):

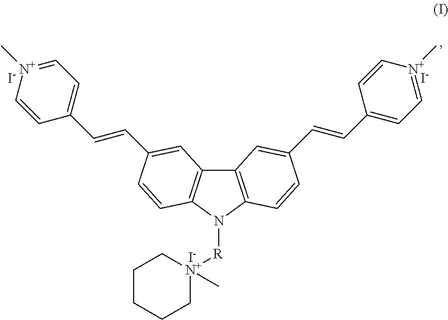

wherein R is —CCOCCOCC— or —CCOCCOCCOCC—;

and a pharmaceutically acceptable carrier.

In a preferred embodiment of the present invention, said pharmaceutical composition further comprise an anti-cancer drug; more preferably, said anti-cancer drug is a telomere- and/or telomerase-targeting agent; even more preferably, said telomere- and/or telomerase-targeting agent is selected from the group consisting of Telomestatin (Tauchi et al., 2003), TMPYP4 (5,10,15,20-tetra(N-methyl-4-pyridyl)porphin) (Grand et al.; 2002), BRACO-19 (9-[4-(N,N-dimethylamino)phenylamino]-3,6-bis(3-pyrrolodino-propionamido) acridine) (Burger et al., 2005), RHPS4 (3,11-difluoro-6,8,13-trimethyl-8H-quino[4,3,2-kl]acridinium methosulfate) (Salvati et al., 2007), CX-3543 (5-fluoro-N-[2-[(2S)-1-methylpyrrolidin-2-yl]ethyl]-3-oxo-6-[3-(pyrazin-2-yl)pyrrolidin-1-yl]-3H-benzo[b]pyrido[3,2,1-kl]phenoxazine-2-carboxamide, also known as quarfloxin) (Drygin et al., 2009), BMVC (3,6-bis(1-methyl-4-vinylpyridinium)carbazole diiodide) (Huang et al., 2008) and combinations thereof.

In a preferred embodiment of the present invention, the R of said compound represented by the formula (I) is —CCOCCOCC— or —CCOCCOCCOCC—; more preferably, is —CCOCCOCCOCC—.

In a preferred embodiment of the present invention, said pharmaceutical composition is used for treating lung cancer, breast cancer, prostate cancer, colon cancer or leukemia; especially, for treating lung cancer.

The present invention also provides a method for treating cancer, comprising administering a therapeutically effective amount of a compound represented by formula (I) to a subject

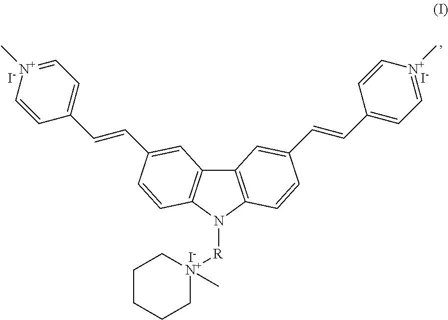

wherein R is —CCOCCOCC— or —CCOCCOCCOCC—.

In a preferred embodiment of the aforesaid method, the compound represented by the formula (I) is administered with an anti-cancer drug; more preferably, said anti-cancer drug is a telomere- and/or telomerase-targeting agent; even more preferably, said telomere- and/or telomerase-targeting agent is selected from the group consisting of Telomestatin, TMPYP4, BRACO-19, RHPS4, CX-3543, BMVC and combinations thereof.

In a preferred embodiment of the aforesaid method, the R of said compound represented by the formula (I) is —CCOCCOCC— or —CCOCCOCCOCC—; more preferably, is —CCOCCOCCOCC—.

In a preferred embodiment of the aforesaid method, the aforesaid method is used for treating lung cancer, breast cancer, prostate cancer, colon cancer or leukemia; more preferably for treating lung cancer.

Yet the present invention provides a method for forming a stabilized G4 structure, comprising: (a) providing a sample comprising chromosome; and (b) contacting an effective amount of a compound represented by formula (I) with said sample

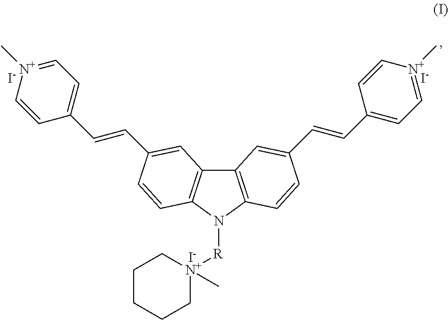

wherein R is —CCOCCOCC— or —CCOCCOCCOCC—.

In a preferred embodiment of the aforesaid method, said compound represented by the formula (I) contacts said sample in a Na⁺ or K⁺ solution; more preferably, in a 5-150 mM K⁺ solution.

In a preferred embodiment of the aforesaid method, said compound represented by the formula (I) contacts the chromosome comprised in said sample; more preferably, contacts the telomere of chromosome comprised in said sample.

In a preferred embodiment of the aforesaid method, said compound represented by the formula (I) contacts said sample in vitro.

In a preferred embodiment of the aforesaid method, said sample is a cancer cell line sample or a clinical sample. The term “clinical sample” means a sample obtained from a patient, including cells obtained from separate tissues, body fluids or excretions of human body.

In a preferred embodiment of the aforesaid method, the R of said compound represented by the formula (I) is —CCOCCOCC— or —CCOCCOCCOCC—; more preferably, —CCOCCOCCOCC—.

The exemplified embodiments of the present invention are described by the following detailed description together with the accompanying drawings. Other features and advantages of the present disclosure may be apparent from the following detailed description and claims.

### BRIEF DESCRIPTION OF THE DRAWINGS

FIG. 1A shows the CD spectra obtained under a salt-deficient condition at room temperature, in which the equivalence ratio of HT24:BMVC-8C3O is from 1:0 to 1:10. FIG. 1B represents a graph of the normalized data of CD intensity at around 265 nm, as a function of the concentration ratio of [BMVC-8C3O]/[HT24].

FIG. 2A represents the CD spectra of the HT24 and its complexes with 1 eq. BMVC-8C3O mixed in a 150 mM K⁺ solution with or without annealing. FIG. 2B represents the CD spectra of the HT24 and its complexes with 1 eq. BMVC-8C3O mixed in a 150 mM Na⁺ solution with or without annealing. FIG. 2C represents the CD spectra of HT24 and its complexes with 5 eq. BMVC-8C3O mixed in a 150 mM K⁺ solution at 37° C. after adding BMVC-8C3O 0.5 h to 24 hr. FIG. 2D represents the ¹H NMR spectra of HT24 and its complexes with 5 eq. BMVC-8C3O mixed in a 150 mM K⁺ solution at 37° C. after adding BMVC-8C3O 2 h and 12 h.

FIG. 3A represents the CD spectra of HT24 in 5 mM K⁺ solution at 25° C. and 95° C. FIG. 3B represents the CD spectra of the complex of HT24 and 5 eq. BMVC-8C3O in 5 mM K⁺ solution at 25° C. and 95° C. FIG. 3C represents the melting temperature curve of HT24 and its complex with 5 eq. BMVC-8C3O in 5 mM or 150 mM K⁺ solution, measured at 265 nm.

FIG. 4A the cell proliferation curve of CL1-0 cancer cells that long-term treated with 1.0 μM BMVC-8C3O. FIG. 4B represents the cell proliferation curve of MRC-5 normal cells that long-term treated with 1.0 μM BMVC-8C30.

### DETAILED DESCRIPTION OF THE PREFERRED EMBODIMENTS

3,6-bis(1-methyl-4-vinylpyridinium)carbazole diiodide (BMVC) is known as a fluorescent marker for cancer diagnosis (see Kang et al., 2007) and a G4 stabilizer for possible anti-tumor agent (see Chang et al., 2004b). BMVC can not only stabilize the G4 structures of human telomeres, but also accelerate telomere shortening and inhibit cancer proliferation (see Huang et al., 2008). In the present invention, the inventors have modified BMVC by substituting the tetraethylene glycol with a methyl-piperidinium cation at N-9 position of BMVC to obtain BMVC-8C3O. It is found that the tetraethylene glycol moiety with a methyl-piperidinium cation can induce the formation of G4 structures and convert the G4 structures of human telomeres from nonparallel to parallel forms in a K⁺ solution, which cannot be achieved by the un-modified BMVC. Moreover, BMVC merely increase the melting temperature of the G4 structures of human telomeres by approximately 20° C., but the BMVC-8C3O of the present invention significantly increases the melting temperature by about 50° C., which is far greater than that of BMVC.

The examples of the present invention are provided hereinafter, however, these examples are not used for limit the scope of the present invention. Those skilled in the art will recognize and understand them without further explanation. All the references are hereby incorporated by reference in its entirety herein.

### EXAMPLES

### Example 1

### Preparation of BMVC derivatives

The synthesis of the target BMVC derivatives were shown in Scheme 1.

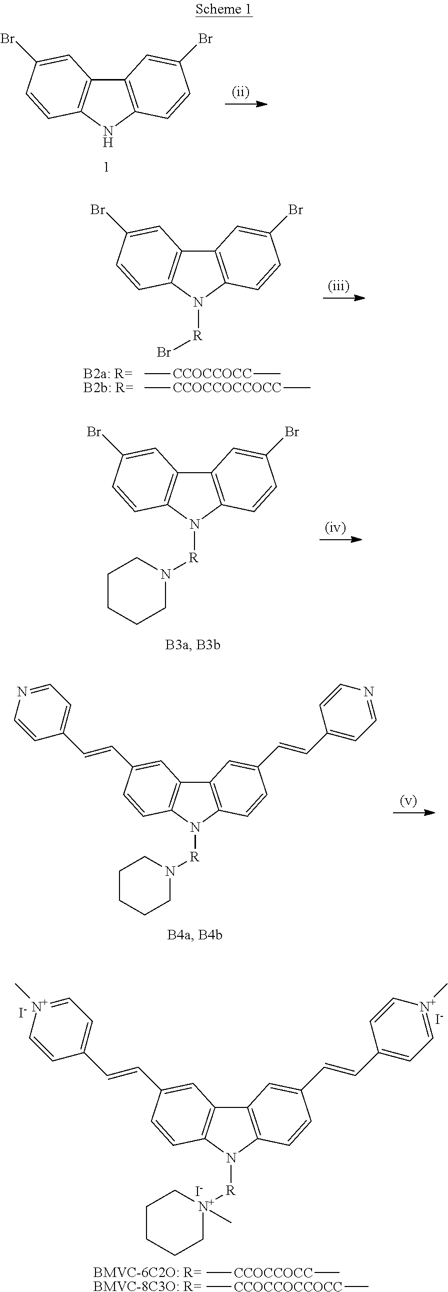

First, compound B2b was synthesized from 3,6-dibromocarbazole 1 (2 g, 6.15 mmole, Aldrich) through 9-position substitution by sodium hydride (0.295 g, 12.3 mmole, Aldrich) in DMF (20 ml) under nitrogen. A dibromo alkane represented by the formula Br—R—Br (R≡CCOCCOCCOCC—) (100 mmole) was then added and the mixture was refluxed for 12 hours. Methanol was slowly added into the reaction system to cool and quench the waste sodium hydride. Then the solution was extracted with H₂O/ethyl acetate (1/1, v/v) twice and the organic layer was dried by MgSO₄. The product B2b was collected and purified via flash column chromatography by silica gel column with hexane/ethyl acetate (2/1, v/v) as the eluent.

The dry powder of compound B2b, piperidine (0.5 ml, Aldrich) and NaH (1.5 mmole) were refluxed in ethanol (20 ml) for 6 hours to obtain the compound B3b which was terminated by piperidine. The solvent was evaporated in vacuum and the residue was purified via flash column chromatography by silica gel column with hexane/ethyl acetate (1/2, v/v) as the eluent to collect the yellow product B3b.

Then the product B3b, 4-vinylpyridine and the mixed powders of Palladium (II) acetate and tri-o-tolylphosphine were dissolved in the triethylamine/acetonitrile solvent pairs and coupled in a high-pressure system, and this system was kept under about 105° C. for two days. The precipitant was collected and then extracted with H₂O/CH₂Cl₂ (1:1, v/v) twice. The solids insoluble in CH₂Cl₂ layer were filtered and collected, washed with hot THF twice, and then dried by MgSO₄. The product was purified by flash column chromatography with CH₂Cl₂/n-hexane (1:1, v/v) as the eluent to obtain the crude powders B4b, which was then added into a 5%-10% triethylamine solution. After that, B4b was refluxed with excess CH₃I in DMF and the target product, N9-substituted BMVC derivatives (BMVC-8C3O), was obtained as an orange-red powder. The yield and NMR information are listed below: 3,6-Bis(1-methyl-4-vinylpyridium iodide)-9-(1-(1-methyl-piperidinium iodide)-3,6, 9-trioxaundecane) carbazole (BMVC-8C3O): (Yield: 86%, mp>300° C.), ¹H NMR (400 MHz, DMSO-d6) δ: 8.80 (d, J=6 Hz, 4H), 8.68 (s, 2H), 8.23 (d, J=16 Hz, 2H), 8.20 (d, J=7.2 Hz, 4H), 7.90 (d, J=8.8 Hz, 2H), 7.76 (d, J=8.4 Hz, 2H), 7.59 (d, J=16 Hz, 2H), 4.64 (t, 2H), 4.24 (s, 6H), 3.82 (t, 2H), 3.71 (t, 2H), 3.47 (m, 4H), 3.38 (m, 10H), 2.97 (s, 3H), 1.67 (m, 4H), 1.43 (m, 2H).

Another N9-substituted BMVC derivative, BMVC-6C20, can be obtained in accordance with the above scheme, but dibromo alkane is different (R═—CCOCCOCC—). The yield and NMR information of BMVC-6C20 are listed below: 3,6-Bis(1-methyl-4-vinylpyridium iodide)-9-(1-(1-methyl-piperidinium iodide)-3, 6-dioxaoctane) carbazole (BMVC-6C20): (Yield: 83%, mp>300° C.), ¹H NMR (400 MHz, DMSO-d6) δ: 8.81 (d, J=6 Hz, 4H), 8.68 (s, 2H), 8.23 (d, J=16 Hz, 2H), 8.21 (d, J=7.2 Hz, 4H), 7.92 (d, J=8.8 Hz, 2H), 7.76 (d, J=8.4 Hz, 2H), 7.58 (d, J=16 Hz, 2H), 4.68 (t, 2H), 4.33 (s, 6H), 3.85 (t, 2H), 3.68 (t, 2H), 3.51 (m, 2H), 3.42 (m, 6H), 2.99 (s, 3H), 1.67 (m, 4H), 1.43 (m, 2H).

### Example 2

### BMVC Derivatives Induce G4 Structure Formation

All oligonucleotides were purchased from Bio Basic Inc. and used without further purification, including the single strand sequence derived from telomere of human chromosome, d(TTAGGG)₄ (HT24) (SEQ ID NO: 1). Solutions of 10 mM Tris-HCl (pH 7.5) and its mixed solutions with KCl were mixed with each DNA sample and heated to 95° C. for 10 min first, cooled slowly to room temperature, and then stored for 48 h at 4° C. before use.

First, circular dichroism spectroscopy (CD spectra) was used to examine whether BMVC-8C3O could induce the formation of G4 structure of human telomere, HT24, under a salt-deficient condition (Chang et al., 2007; Bugaut et al., 2008; Giraldo et al., 1994).

It is well-known that the linear parallel G4 structures, such as a propeller form, give a positive band at around 265 nm and a negative band at around 240 nm, while the anti-parallel G4 structures, such as a basket or chair form, show two positive bands at around 295 nm and 240 nm and a negative band at around 265 nm. In addition, the hybrid type G4 structures (3+1, including 3 parallel and 1 anti-parallel G4 structures) give a positive CD band at around 290 nm and a positive shoulder band at around 265 nm. The anti-parallel and hybrid type are both so-called “non-parallel” G4 structures. These spectral features are mainly attributed to the specific guanine stacking in various G4 structures.

J-815 spectropolarimeter (Jasco, Japan) with a 2 nm bandwidth at a 50 nm/min scan speed and a 0.2 nm step resolution was used to obtain CD spectra, and the following data was provided as an average of 10 scan results. The CD spectra were measured by monitoring the G4 structures under N₂ over the range of 210 nm to 350 nm, and the thermal melting curves, as a function of temperature, were obtained by monitoring the CD intensity at 265 nm. Three independently scans were recorded for each sample. The melting temperature (Tm) was measured from the first differentiation of the melting curve.

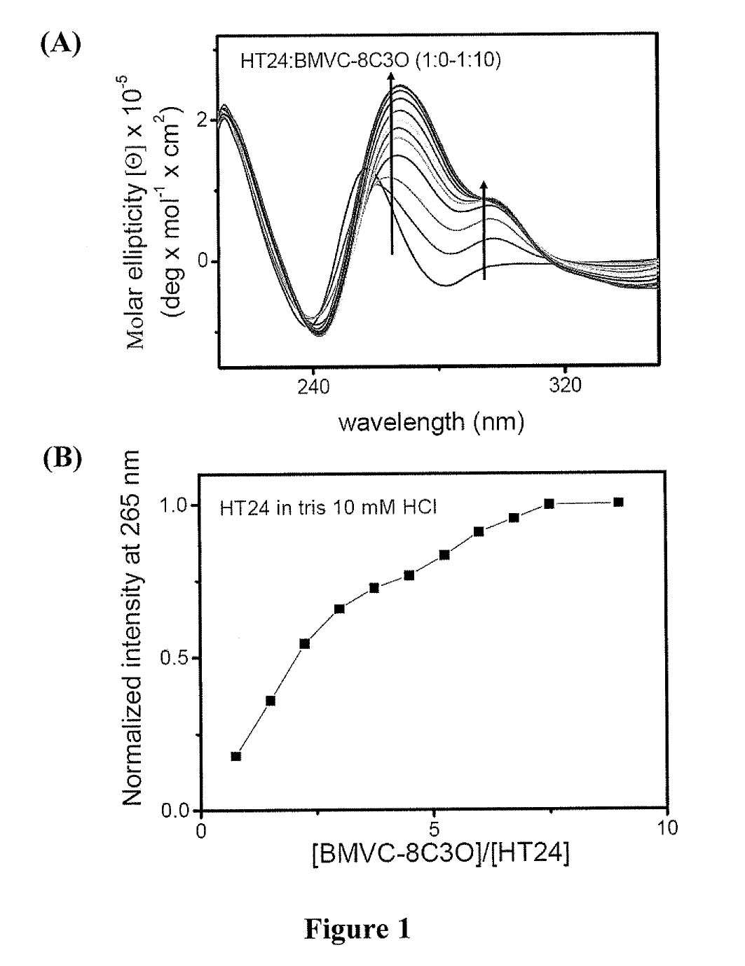

FIG. 1A shows the CD spectra of 20 μM HT24 obtained under a salt-deficient condition at room temperature. This function graph is obtained by titrating 20 μM HT24 with BMVC-8C3O, in which the equivalence ratio of HT24:BMVC-8C3O is from 1:0 to 1:10. Upon this titration, the CD band at around 295 nm gradually increases, which shows the anti-parallel G4 structures gradually increase. The CD band at 295 nm increases until the equivalence ratio of HT24:BMVC-8C3O (G4 ligand) reaches 1:3. Meanwhile, the CD band at around 265 nm gradually increases during the titration until the equivalence ratio of HT24:BMVC-8C3O reaches 1:10, which shows the parallel G4 structures also gradually increase. At last, a hybrid of parallel and anti-parallel G4 structures is formed. These data show that BMVC-8C3O can induce G4 formation under a salt-deficient condition at room temperature, and the parallel and anti-parallel G4 structures possibly coexist under a higher concentration of BMVC-8C30.

In addition, FIG. 1B, a function graph of BMVC-8C3O concentration, shows the normalized data of CD intensity at around 265 nm. When the concentration of BMVC-8C3O is higher, the CD intensity at 265 nm is stronger, which means that BMVC-8C3O induces the formation of parallel G4 structures.

From above, it should be clear that HT24 itself does not have any G4 structure, and the addition of BMVC-8C3O induces HT24 to form G4 structures which is a hybrid of parallel and anti-parallel G4 structures.

### Example 3

### BMVC Derivatives Convert G4 Structure Conformation

5 μM HT24 and 1 eq. BMVC-8C3O (the equivalence ratio of HT24:BMVC-8C30 is 1:1) were used to detect whether BMVC-8C3O could convert the G4 structure conformation of HT24 from non-parallel to parallel.

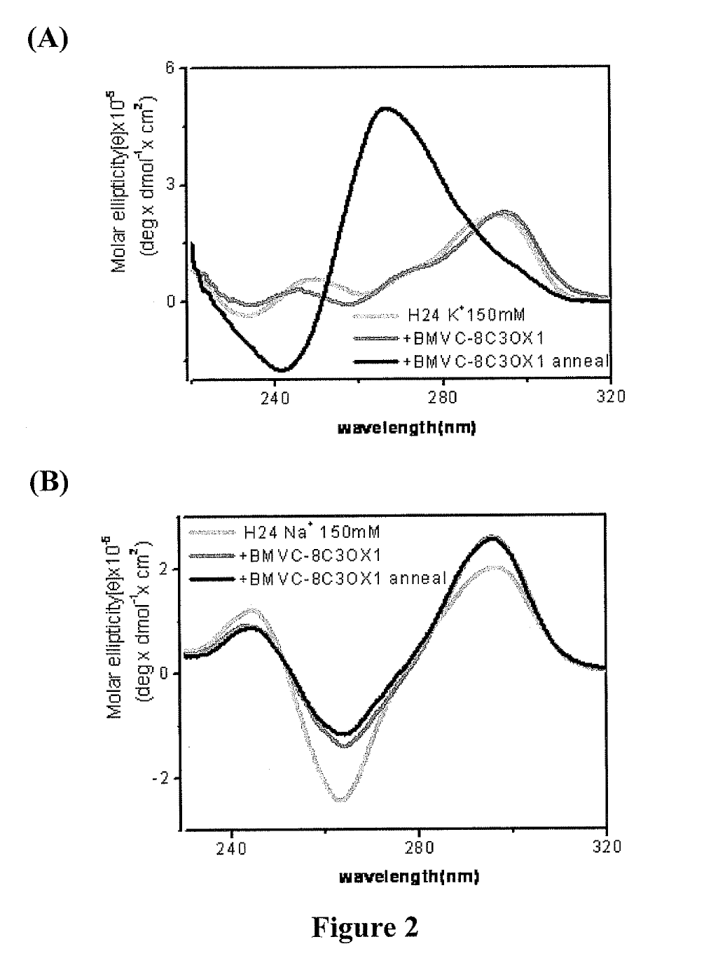

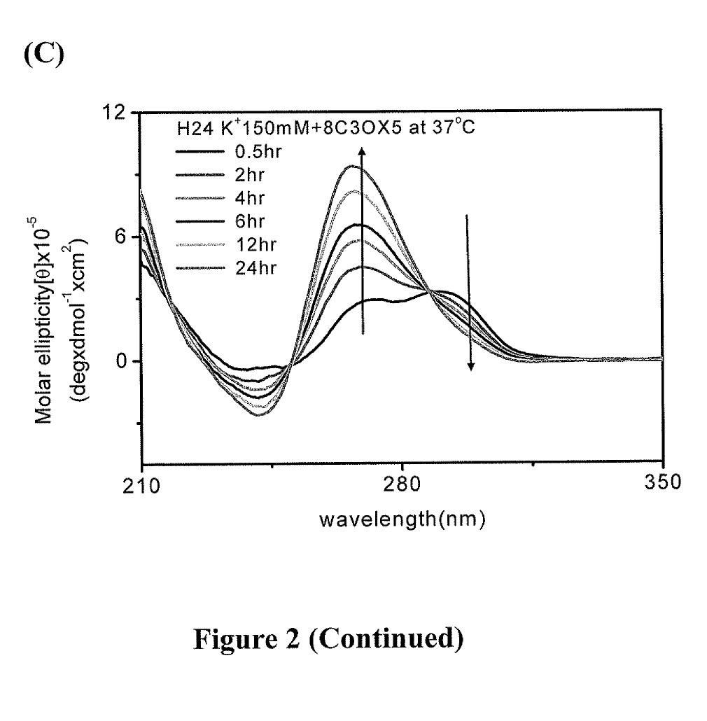

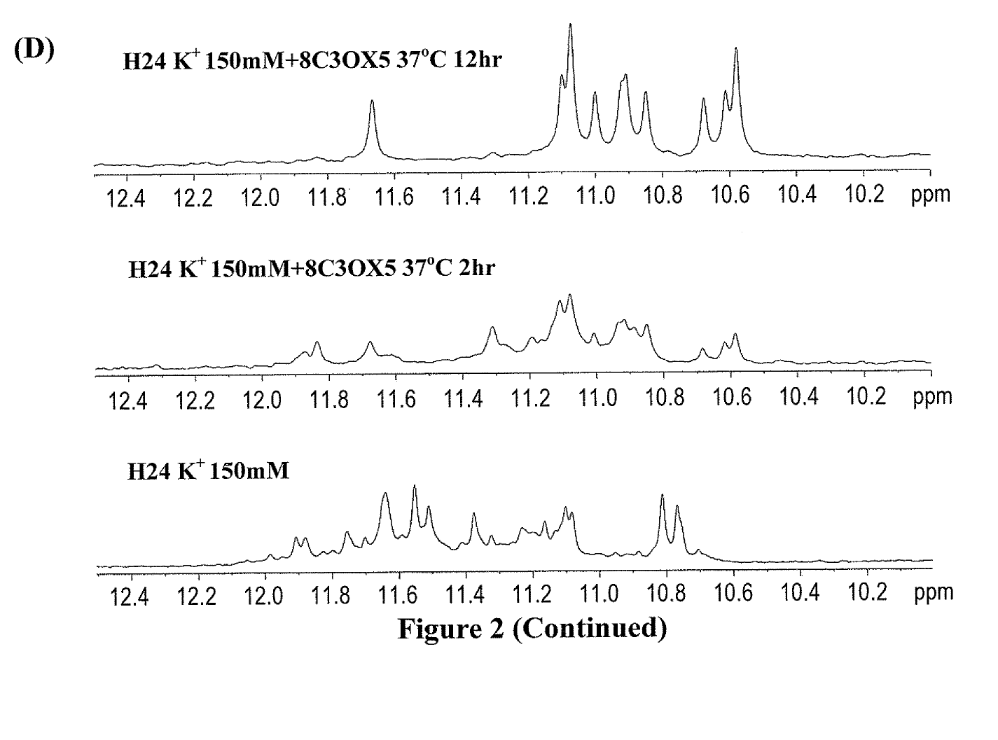

The CD spectra of the HT24 and its complexes with 1 eq. BMVC-8C3O mixed in a 150 mM K⁺ solution or in a 150 mM Na⁺ solution were measured right after the mixing step under room temperature, as shown in FIGS. 2A and 2B (+BMVC-8C3O×1). Another set of spectra were obtained after these samples were annealed, i.e. heated at 95° C., and then gradually cooled down to room temperature (+BMVC-8C3O×1 anneal). After comparing the CD spectra before and after annealing, it has been found that the CD pattern shows significant spectral changes in K⁺ solution before and after annealing, but in Na⁺ solution, only CD intensity changes are observed. These spectral features are consistent with the spectral changes of d[G₃(T₂AG₃)₃] (HT21) in K⁺ or Na⁺ solution upon the addition of 40% PEG (see Xue et al., 2007). That is to say, the peak at 265 nm (parallel G4 structure) increases and the peak at 290 nm (non-parallel G4 structure) decreases. This is, BMVC-8C3O and 40% PEG can converse the G4 structures from the non-parallel to the parallel.

FIG. 2C shows the spectral changes upon addition of 5 eq. BMVC-8C3O at 37° C., as a function of time. The CD results suggest that BMVC-8C3O can convert the conformation of G4 structures from non-parallel to parallel at 37° C. in K⁺ solution without annealing.

In addition, 0.1 mM HT24 was mixed with 5 eq. BMVC-8C3O and dissolved in a H₂O/D₂O (90%/10%) solution containing 10 mM tris-HCl (pH 7.5) and 150 mM KCl to prepare samples for NMR (control sample was without BMVC-8C3O). Then the imino protons in the chemical shift range of 9-14 ppm were measured on a Bruker AVIII 800 MHz spectrometer using a pulsed-gradient spin-echo sequence with a selective refocusing pulse, and the chemical shift was measured relative to a D₂O solution of DSS as an external reference. FIG. 2D shows the imino proton spectra (i.e. ¹H NMR spectra) of HT24 and its complex with BMVC-8C3O in 150 mM K⁺ solution after adding BMVC-8C3O 2 h and 12 h. The NMR spectra show significant changes after adding 5 eq. BMVC-8C3O, which indicate that BMVC-8C3O can induce the structural change of HT24 in K⁺ solution.

When 1 eq. BMVC-8C3O is used, the spectra changes are only induced after annealing. However, when the concentration of BMVC-8C3O is increasing, the necessity of the annealing step decreases. For example, 5 eq. BMVC-8C3O induces spectra changes of the complex of HT24 and BMVC-8C3O in 150 mM K⁺ solution at 37° C. (converting from non-parallel to parallel), and the annealing step is not necessary (see FIG. 2C).

From the data shown in FIGS. 1 and 2, it should be clear that the BMVC-8C3O of the present invention induces parallel and non-parallel G4 structures of HT24 in a salt-deficient condition (i.e. without Na⁺ or K⁺ ions). Yet, in a condition with K⁺ ions, the BMVC-8C3O of the present invention converts G4 structure of HT24 from non-parallel to parallel.

### Example 4

### BMVC Derivatives Stabilize G4 Structures

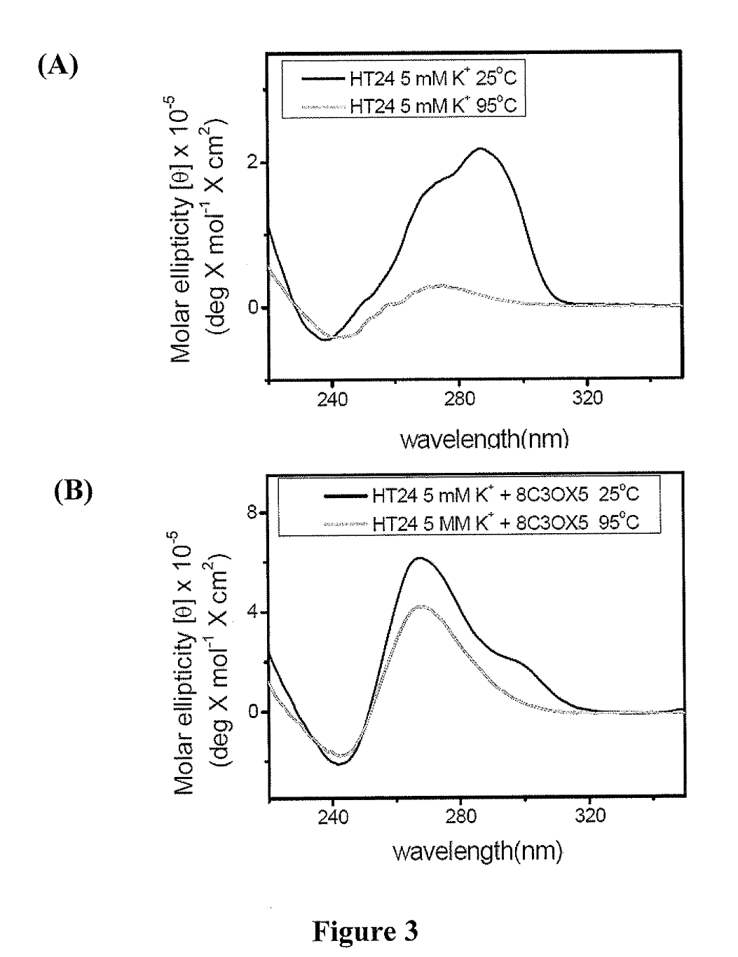

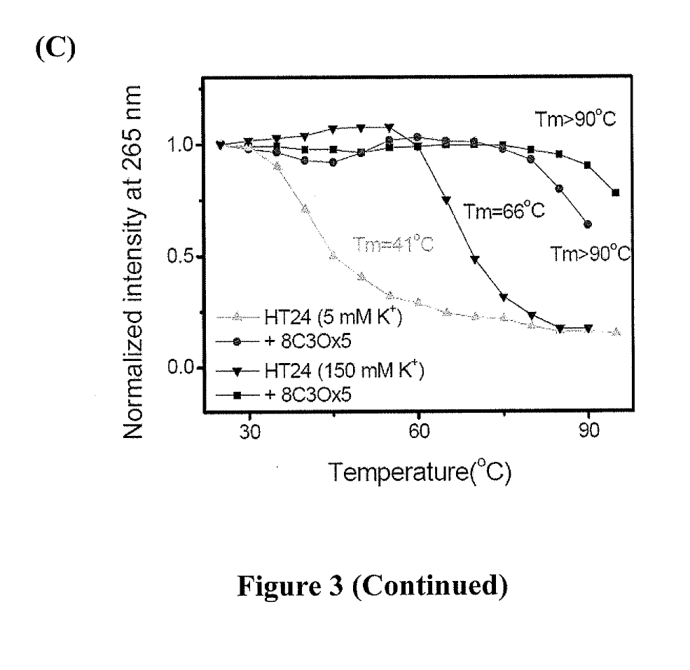

The BMVC-8C3O of the present invention can not only induce the formation of G4 structures, but also stabilize G4 structures. FIGS. 3A and 3B show the CD spectra of HT24 and its complex with 5 eq. BMVC-8C3O in 5 mM K⁺ solution at 25° C. and 95° C., respectively. FIG. 3C shows the normalized CD intensity of HT24 and its complex with 5 eq. BMVC-8C3O in 5 mM or 150 mM K⁺ solution at 265 nm, as a function graph of temperature. After adding 5 eq. BMVC-8C3O at 37° C. 24 hours, the melting temperature (Tm) of HT24 in 5 mM K⁺ solution is significantly increased from approximately 41° C. to >90° C., and the temperature is enhanced by about 50° C. This shows that BMVC-8C3O is a better G4 stabilizer, and can be used as an anti-tumor agent.

### Example 5

### BMVC Derivatives Slow Cancer Cell Proliferation

CL1-0 lung cancer cells and MRC-5 normal human lung fibroblasts were used in the following experiments, wherein CL1-0 cells were cultured in RPMI-1640 medium containing 10% FBS (fetal bovine serum) and 1% antibiotics (including penicillin and streptomycin), and MRC-5 cells were cultured in MEM medium containing 10% FBS and 1% antibiotics.

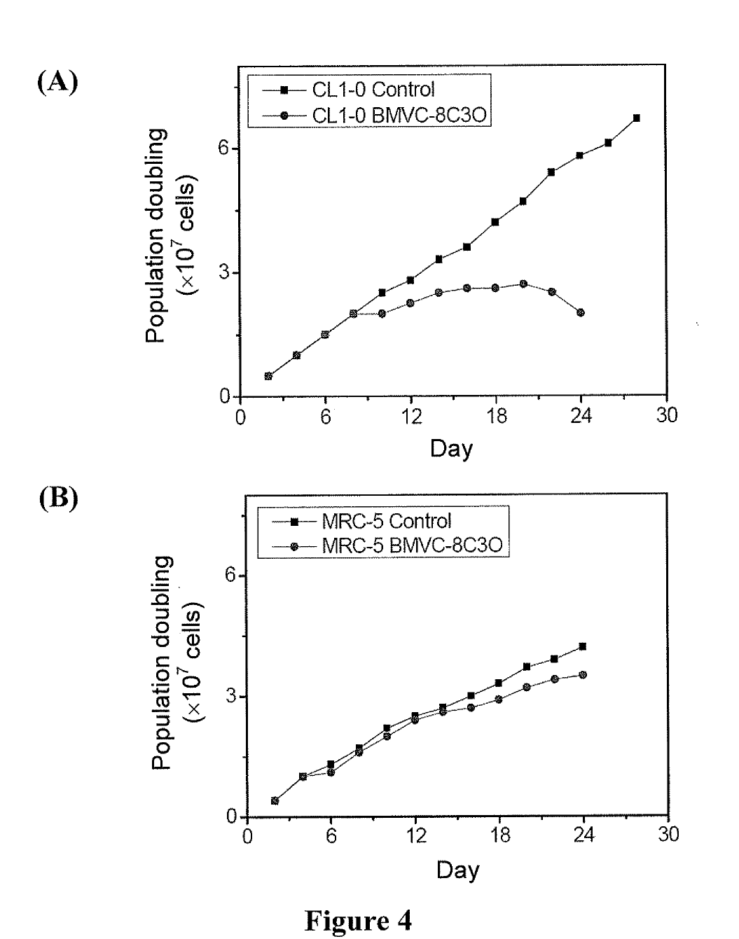

When these cells were subcultured, culture medium containing 1.0 μM BMVC-8C3O (1% DMSO for the control) was used to separate these cells into 5×10⁵ cells/petri dish having a diameter of 6 cm. After 2 or 3 days, these cells were trypsinized and counted, and subcultured again with the same density and method. Cells were subcultured repeatedly until the total cell number was less than 5×10⁵. The result is shown in FIG. 4.

FIGS. 4A and 4B show the cell proliferation curves of CL1-0 cancer cells and MRC-5 normal cells that long-term treated with 1.0 μM BMVC-8C3O. From FIG. 4A, it is obvious that the proliferation of CL1-0 cancer cells is slowed from around day 10, and stopped at around day 20. FIG. 4B shows that BMVC-8C3O does not significantly affect MRC-5 normal cells. From above, it shows that BMVC-8C3O is an excellent G4 stabilizer and a good candidate for inhibiting proliferation of cancer cells.

The present invention has taken advantage of the molecular crowding effect to modify G4 ligands, thereby inducing G4 structural changes of HT24 at 37° C. Particularly, the BMVC derivatives (such as 3 eq. or more of BMVC-8C3O) induce an extremely stable propeller G4 structure, enhance the melting temperature by approximately 50° C., and stop proliferation of cancer cells (such as CL1-0 cells) without affecting normal cells (such as MRC-5 cells). Furthermore, similar results are also observed in the case of BMVC-6C20 (data not shown). The present invention provides a better G4 stabilizer and the anti-cancer application thereof.

The preferred examples of the present invention are disclosed herein; however, these examples are not used for limiting the scope of the present invention. The amendments and modifications can be made by those skilled in the art without departing the spirit and scope of the present invention.

### REFERENCES

Ambrus, A., Chen, D., Dai, J. X., Bialis, T., Jones, R. A., Yang, D., Nucleic Acids Res. 2006, 34, 2723-2735.
Blackburn, E. H. and Greider, C. W., Telomeres, 1996, Cold Spring Harbor Laboratory Press, New York.
Blasco, M. A., Nat. Rev. Genet. 2005, 6, 611-622.
Bodnar, A. G., Ouellette, M., Frolkis, M., Holt, S. E., Chiu, C. P., Morin, G. B., Harley, C. B., Shay, J. W., Lichtsteiner, S., Wright, W. E., Science 1998, 279, 349-352.
Bugaut, A., Balasubramanian, S., Biochemistry 2008, 47, 689-697.
Burger, A. M.; Dai, F.; Schultes, C. M.; Reszka, A. P.; Moore, M. J.; Double, J. A.; Neidle, S. Cancer Res 2005, 65, 1489.
Cech, T. R., Cell 2004, 116, 273-279.
Chang, C. C., Kuo, I-C., Ling, I-F., Chen, C. T., Chen, H. C., Lou, P. J., Lin, J. J. and Chang, T.-C., Anal. Chem. 2004a, 76, 4490-4494.
Chang, C. C., Kuo, I-C., Lin, J. J., Lu, Y. C., Chen, C. T., Back, H. T., Lou, P. J., Chang, T.-C., Chem. Biodivers. 2004b, 1, 1377-1384.
Chang, C. C., Chien, C. W., Lin, Y. H., Kang, C. C., Chang, T.-C., Nucleic Acids Res. 2007, 35, 2846-2860.
Collie, G. W., Haider, S. M., Neidle, S., Parkinson, G. N., Nucleic Acids Res. 2010, 38, 5569-5580.
DeCian, A., DeLemos, E., Mergny, J.-L., Teulade-Fichou, M.-P., Monchaud, D., J. Am. Chem. Soc. 2007, 129, 1856-1857.
DeCian, A., Lacroix, L., Douarre, C., Temime-Smaali, N., Trentesaux, C., Riou, J. F., Mergny, J. L., Biochimie 2008, 90, 131-155.
Drygin, D.; Siddiqui-Jain, A.; O'Brien, S.; Schwaebe, M.; Lin, A.; Bliesath, J.; Ho, C. B.; Proffitt, C.; Trent, K.; Whitten, J. P.; Lim, J. K.; Von Hoff, D.; Anderes, K.; Rice, W. G. Cancer Res 2009, 69, 7653.
Folini, M.; Venturini, L.; Cimino-Reale, G.; Zaffaroni, N. Expert Opin. Ther. Targets, online, 2011.
Giraldo, R., Suzuki, M., Chapman, L., Rhodes, D., Proc. Natl. Acad. Sci. USA, 1994, 91, 7658-7662.
Grand, C. L.; Han, H.; Munoz, R. M.; Weitman, S.; Von Hoff, D. D.; Hurley, L. H.; Bearss, D. J. Mol Cancer Ther 2002, 1, 565.
Huang, F. C., Chang, C. C., Lou, P. J., Kuo, I-C., Chien, C. W., Chen, C. T., Shieh, F. Y., Chang, T.-C., Lin, J. J., Mol. Cancer. Res. 2008, 6, 955-964.
Hurley, L. H., Nature Rev. Cancer 2002, 2, 188-200.
Kan, Z.-Y., Yao, Y., Wang, P., Li, X.-H., Hao, Y.-H., Tan, Z., Angew. Chem. Int. Ed. Engl. 2006, 45, 1629-1632.
Kang, C. C., Chang, C. C., Chang, T.-C., Liao, L. J., Lou, P. J., Xie, W., Yeung, E. S., Analyst 2007, 132, 745-749.
Lim, K. W., Amrane, S., Bouaziz, S., Xu, W., Mu, Y., Patel, D. J., Luu, K. N., Phan, A. T., J. Am. Chem. Soc. 2009, 131, 4301-4309.
Luu, K. N., Phan, A. T., Kuryavyi, V., Lacroix, L., Patel, D. J., J. Am. Chem. Soc. 2006, 128, 9963-9970.
Maizels, N., Nat. Struct. Mol. Biol. 2006, 13, 1055-1059.
Martadinata, H., Phan, A. T., J. Am. Chem. Soc. 2009, 131, 2570-2578.
Mergny J. L., and Hélène, C., Nature Med. 1998, 4, 1366-1367.
Miyoshi, D., Nakao, A., Sugimoto, N., Biochemistry 2002, 41, 15017-15024.
Miyoshi, D., Karimata, H., Sugimoto, N., J. Am. Chem. Soc. 2006, 128, 7957-7963.
Miyoshi, D., Sugimoto, N., Biochimie 2008, 90, 1040-1051.
Monchaud, D., Teulade-Fichou, M.-P., Org. Biomol. Chem. 2008, 6, 627-636.
Müller, S., Panto [symbol] , G. D., Rodriguez, R., Balasubramanian, S., Chem. Commun. 2009, 80-82.
Neidle S., and Parkinson, G., Nature Rev. Drug Discov. 2003, 1, 383-393.
Parkinson, G. N., Lee, M. P. H., Neidle, S., Nature 2002, 417, 876-880.
Reed, J. E., Arnal, A. A., Neidle, S., Vilar, R., J. Am. Chem. Soc. 2006, 128, 5992-5993.
Salvati, E.; Leonetti, C.; Rizzo, A.; Scarsella, M.; Mottolese, M.; Galati, R.; Sperduti, I.; Stevens, M. F.; D'Incalci, M.; Blasco, M.; Chiorino, G.; Bauwens, S.; Horard, B.; Gilson, E.; Stoppacciaro, A.; Zupi, G.; Biroccio, A. J Clin Invest 2007, 117, 3236.
Tauchi, T.; Shin-Ya, K.; Sashida, G.; Sumi, M.; Nakajima, A.; Shimamoto, T.; Ohyashiki, J. H.; Ohyashiki, K. Oncogene 2003, 22, 5338.
Wang Y., and Patel, D. J., Structure 1993, 1, 262-283.
Williamson, J. R., Annu. Rev. Biophys. Biomol. Struct. 1994, 23, 703-730.
Xue, Y., Kan, Z. Y., Wang, Q., Yao, Y., Liu, J., Hao, Y. H., Tan, Z., J. Am. Chem. Soc. 2007, 129, 11185-11191.
Zahler, A. M., Williamson, J. R., Cech, T. R., Prescott, D. M., Nature 1991, 350, 718-720.
Zhou, J., Wei, C., Jia, G., Wang, X., Tang, Q., Feng, Z., Li, C., Biophys. Chem. 2008, 136, 124-127.

1. A pharmaceutical composition for treating cancer, comprising a therapeutically effective amount of a compound represented by formula (I): 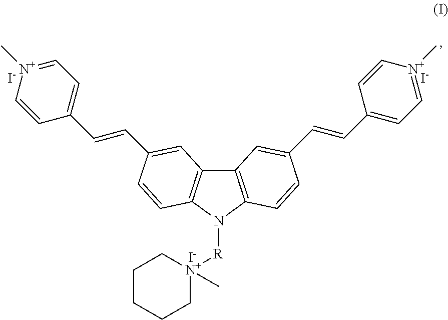
wherein R is —CCOCCOCC— or —CCOCCOCCOCC—;
and a pharmaceutically acceptable carrier.

2. The pharmaceutical composition according to claim 1, further comprising an anti-cancer drug.

3. The pharmaceutical composition according to claim 2, wherein said anti-cancer drug is a telomere- and/or telomerase-targeting agent.

4. The pharmaceutical composition according to claim 3, wherein said telomere- and/or telomerase-targeting agent is selected from the group consisting of Telomestatin, TMPYP4, BRACO-19, RHPS4, CX-3543, BMVC and combinations thereof.

5. The pharmaceutical composition according to claim 1, wherein the R of said compound represented by the formula (I) is —CCOCCOCC—.

6. The pharmaceutical composition according to claim 1, wherein the R of said compound represented by the formula (I) is —CCOCCOCCOCC—.

7. The pharmaceutical composition according to claim 1, which is used for treating lung cancer, breast cancer, prostate cancer, colon cancer or leukemia.

8. The pharmaceutical composition according to claim 7, which is used for treating lung cancer.

9. A method for treating cancer, comprising administering a therapeutically effective amount of a compound represented by formula (I) to a subject 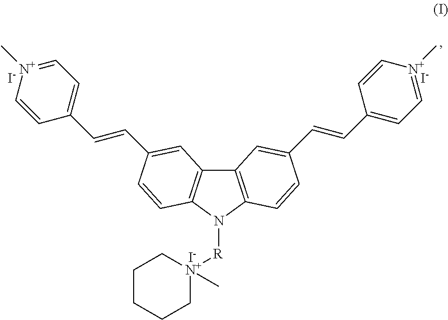
wherein R is —CCOCCOCC— or —CCOCCOCCOCC—.

10. The method according to claim 9, wherein said compound represented by the formula (I) is administered with an anti-cancer drug.

11. The method according to claim 10, wherein said anti-cancer drug is a telomere- and/or telomerase-targeting agent.

12. The method according to claim 11, wherein said telomere- and/or telomerase-targeting agent is selected from the group consisting of Telomestatin, TMPYP4, BRACO-19, RHPS4, CX-3543, BMVC and combinations thereof.

13. The method according to claim 9, wherein the R of said compound represented by the formula (I) is —CCOCCOCC—.

14. The method according to claim 9, wherein the R of said compound represented by the formula (I) is —CCOCCOCCOCC—.

15. The method according to claim 9, which is used for treating lung cancer, breast cancer, prostate cancer, colon cancer or leukemia.

16. The method according to claim 15, which is used for treating lung cancer.

17. A method for forming a stabilized G-quadruplex structure, comprising:
(a) providing a sample comprising chromosome; and
(b) contacting an effective amount of a compound represented by formula (I) with said sample 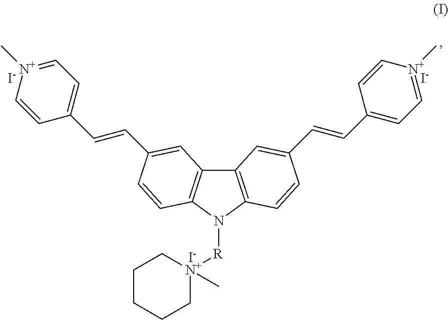
wherein R is —CCOCCOCC— or —CCOCCOCCOCC—.

18. The method according to claim 17, wherein said compound represented by the formula (I) contacts said sample in a Na⁺ or K⁺ solution.

19. The method according to claim 17, wherein said compound represented by the formula (I) contacts the chromosome comprised in said sample.

20. The method according to claim 19, wherein said compound represented by the formula (I) contacts the telomere of chromosome comprised in said sample.

21. The method according to claim 17, wherein said compound represented by the formula (I) contacts said sample in vitro.

22. The method according to claim 17, wherein said sample is a cancer cell line sample or a clinical sample.

23. The method according to claim 17, wherein the R of said compound represented by the formula (I) is —CCOCCOCC—.

24. The method according to claim 17, wherein the R of said compound represented by the formula (I) is —CCOCCOCCOCC—.
